(19) United States
(12) Patent Application Publication
(10) Pub. No.: US 2008/0167319 A1
(43) Pub. Date: Jul. 10, 2008

(54) 7-(4-(4-[3-CHLORO-2-(TRIFLUOROMETHYL)PHENYL]PIPERAZIN-1-YL)BUTOXY)-[1,8]-NAPHTHYRIDIN-2(1H)-ONE

(75) Inventors: David Alan Favor, Sandwich (GB); Joseph Thomas Repine, Ann Arbor, MI (US)

Correspondence Address: PFIZER INC;Steve T. Zelson, 150 EAST 42ND STREET, 5TH FLOOR - STOP 49, NEW YORK, NY, 10017-5612, US

(73) Assignee: Pfizer Inc.

(21) Appl. No.: 11/968,231

(22) Filed: Jan. 2, 2008

Related U.S. Application Data
(60) Provisional application No. 60/883,447, filed on Jan. 4, 2007.

(51) Int. Cl. A61K 31/497; C07D 403/14; A61P 25/00

(52) U.S. Cl. 514/253.04; 544/362

(57) ABSTRACT

This invention relates to a compound of formula (I)

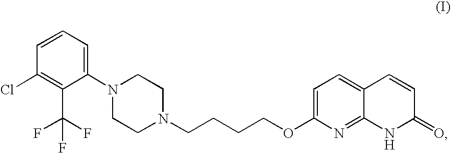

or pharmaceutically acceptable salts thereof, pharmaceutical compositions containing the compound or a salt thereof, and its use as a medicament for the treatment of schizophrenia, bipolar disorder, or other central nervous system disorders.

### CROSS REFERENCE TO RELATED APPLICATION

The present invention claims benefit of U.S. Ser. No. 60/883,447 filed on Jan. 4, 2007, which is hereby incorporated by reference in its entirety.

### BACKGROUND OF THE INVENTION

This invention relates to a novel compound that has activities at various receptors found in the central nervous system (CNS) and is useful for treating Schizophrenia or other CNS disorders.

US Patent Application Publication No.: US 2005/0043309 A1 and International Application Publication No. WO 2005/019215 disclose compounds that are [1,8]naphthyridin-2-one derivatives, methods of making the compounds, pharmaceutical compositions containing the compounds, and their use for the treatment of schizophrenia and other central nervous system (CNS). The disclosure of US 2005/0043309 A1 and WO 2005/019215 generically encompasses the compound 7-(4-{4-[3-chloro-2-(trifluoromethyl)phenyl]piperazin-1-yl}butoxy)-[1,8]-naphthyridin-2(1H)-one; but neither document exemplifies, specifically mentions, or otherwise specifically identifies this compound.

### SUMMARY OF THE INVENTION

In one aspect, the present invention provides a compound of formula (I),

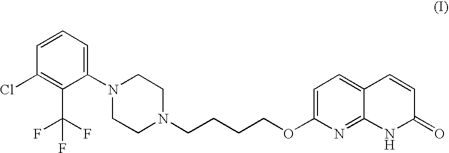

and pharmaceutically acceptable salts thereof.

In another aspect, the present invention provides a composition comprising a compound of formula (I) or a pharmaceutically acceptable salt thereof, and a pharmaceutically acceptable excipient.

In still another aspect, the present invention provides a method of treating a CNS disorder in a mammal, comprising administering to the mammal a therapeutically effective amount of a compound of formula (I) or a pharmaceutically acceptable salt thereof.

Additional aspects of the inventions are disclosed in the following description, including examples and the claims.

### DETAILED DESCRIPTION OF THE INVENTION

In one aspect, the present invention provides a compound of formula (I) and pharmaceutically acceptable salts thereof. The compound of formula (I) is also known by chemical name 7-{4-[4-(3-Chloro-2-trifluoromethyl-phenyl)-piperazin-1-yl]-butoxy}-1H-[1,8]naphthyridin-2-one, or 7-(4-{4-[3-chloro-2-(trifluoromethyl)phenyl]piperazine-1-yl}butoxy)-[1,8]-naphthyridin-2(1H)-one (IUPAC name). The compound can be prepared by the process described in Examples 1A or 1B.

The present invention also encompasses isotopically-labeled compound of formula (I), wherein one or more atoms are replaced by atoms having the same atomic number, but an atomic mass or mass number different from the atomic mass or mass number which predominates in nature. Examples of isotopes suitable for inclusion in the compounds of formula (I) include isotopes of hydrogen, such as ²H and ³H, carbon, such as ¹¹C, ¹³C and ¹⁴C, chlorine, such as ³⁸Cl, fluorine, such as ¹⁸F, iodine, such as ¹²³I and ¹²⁵I, nitrogen, such as ¹³N and ¹⁵N, oxygen such as ¹⁶O, ¹⁷O and ¹⁶O, phosphorus, such as ³²P, and sulfur, such as ³⁵S. Certain isotopically-labelled compounds of formula (I) for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known in the art.

Pharmaceutically acceptable salts of the compound of formula (I) includes salts derived from either inorganic acid or organic acid. The salts may be made by conventional methods known in the art. Examples of inorganic acids include hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like. Examples of organic acids include 1-hydroxy-2-naphthoic, 2-napsylatic, 3-hydroxy-2-naphthoic, acetic, adipic, ascorbic, aspartic, benzenesulfonic, benzoic, besylic, camsylic, cholic, citric, D- and L-lactic, D and L-tartric, edisylic, estolic, fumaric, galacturonic, gluceptic, gluconic, glucuronic, glutamic, hibenzic, hippuric, isethionic, isobutyric, lactobionic, malic, maleic, malonic, mandelic, methanesulfonic, mucic, napadisylic, nicotinic, oleic, orotic, oxalic, pamoic, phthalic, propionic, p-tolylsulfonic, saccharic, salicylic, stearic, suberic, succinic, sulphosalicylic, tryptophanic, and amino acids. Examples of particular pharmaceutically acceptable salts of the compound of formula (I) include salts derived from hydrochloric acid and phosphoric acid.

The compound of formula (I) and pharmaceutical acceptable salts thereof are useful as pharmaceutical agents for the treatment of a central nervous system disorder. Thus, in another aspect, the present invention provides a method of treating a central nervous system disorder in a mammal, comprising administering to the mammal a therapeutically effective amount of a compound of formula (I) or a pharmaceutically acceptable salt thereof.

As used herein, the term “treat,” “treating,” or “treatment,” in all grammatical forms, means (a) alleviating, reducing the severity of, slowing the onset of, or eliminating one or more symptoms associated with the disorder; (b) causing regression or delaying the progression of the disorder; (c) stabilizing (i.e., not worsening) the state of the disorder; or (d) preventing the occurrence or recurrence of the disorder.

As used herein, the term “therapeutically effective amount” refers to an amount of the compound of formula (I) or a pharmaceutically acceptable salt thereof that, when administered, is sufficient to treat a disorder intended to be treated, wherein “treat” is as defined herein.

As used herein, the term “mammal” means a member of the class of vertebrate animals characterized by the presence of mammary glands, the presence of hair or fur, and warm-blooded bodies. Examples of mammals includes: humans; companion animals such as cats and dogs; non-human primates such as monkeys and chimpanzees; livestock such as horses, cows, pigs, and sheep; and rodents such as rats, mice, guinea pigs, rabbits, hamsters, and transgenic mice.

As used herein, the term “central nervous system disorder” (“CNS disorder”) means a neurological disorder that affects the brain or spinal cord. Examples of CNS disorders that may be treated by a method of the invention include: single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma. Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremor, chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy.

In a particular embodiment, the present invention provides a method of treating Schizophrenia or bipolar disorder in a human, comprising administering to the human a therapeutically effective amount of a compound of formula (I) or a pharmaceutically acceptable salt thereof.

The therapeutically effective amount of a compound of the invention for treating a particular CNS disorder described herein above can be determined in a variety of ways known in the art. For example, an effective amount can be determined by administering various amounts of the compound or salt thereof to an animal having a particular condition and then determining the effect on the animal. In general, the compound or salt thereof may be administered orally at doses ranging from about 0.03 to about 10 mg per kg of body weight. For an adult human patient having a body weight of about 70 kg, the compound or salt thereof can be administered daily at a dosage range from about 3 mg to about 150 mg, and typically from 10 mg to about 100 mg, in single or divided doses (i.e., from 1 to 4 doses per day). The specific dose levels for any particular patient may vary and will depend upon a variety of factors known to a person skilled in the art. Examples of such factors include: the results of clinical studies; the time, frequency, and route of administration; the particular mammal being treated, the patient's age, sex, weight, and general health condition, the type and severity of the disorder being treated; and the use of other medications, if any, by the patient. Frequency of dosage may also vary depending on some of the above factors.

For the treatment of depression, anxiety, schizophrenia, bipolar, or any other CNS disorders referred to above, a compound of this invention can be used in conjunction or combination with one or more other therapeutic agents. Thus, in a further aspect, the present invention provides a method of treating a CNS disorder described herein above in a mammal, comprising administering to the mammal:

(a) a compound of formula (I), or a pharmaceutically acceptable salt thereof; and

(b) an additional therapeutic agent.

As used herein, the term “additional therapeutic agent” refers to any therapeutic agent, other than the compound of formula (I), or salt thereof, that is useful for the treatment of a subject disorder. Examples of additional therapeutic agents include antidepressants and anti-anxiety agents. Examples of particular classes of antidepressants that can be used in combination with the compounds of the invention include norepinephrine reuptake inhibitors, selective serotonin reuptake inhibitors (SSRIs), NK-1 receptor antagonists, monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, and atypical antidepressants. Suitable norepinephrine reuptake inhibitors include tertiary amine tricyclics and secondary amine tricyclics. Examples of suitable tertiary amine tricycles and secondary amine tricyclics include amitriptyline, clomipramine, doxepin, imipramine, trimipramine, dothiepin, butripyline, iprindole, lofepramine, nortriptyline, protriptyline, amoxapine, desipramine and maprotiline. Examples of suitable selective serotonin reuptake inhibitors include fluoxetine, fluvoxamine, paroxetine, and sertraline. Examples of monoamine oxidase inhibitors include isocarboxazid, phenelzine, and tranylcyclopramine. Examples of suitable reversible inhibitors of monoamine oxidase include moclobemide. Example of suitable serotonin and noradrenaline reuptake inhibitors of use in the present invention include venlafaxine. Examples of suitable CRF antagonists include those compounds described in International Patent Application Nos. WO 94/13643, WO 94/13644, WO 94/13661, WO 94/13676 and WO 94/13677. Examples of suitable atypical anti-depressants include bupropion, lithium, nefazodone, trazodone and viloxazine. Examples of suitable NK-1 receptor antagonists include those disclosed in WO 01/77100. Examples of suitable classes of anti-anxiety agents that can be used in combination with the compounds of the invention include benzodiazepines and serotonin 1A (5-HT_{1A}) agonists or antagonists, especially 5-HT_{1A} partial agonists, and corticotropin releasing factor (CRF) antagonists. Suitable benzodiazepines include alprazolam, chlordiazepoxide, clonazepam, chlorazepate, diazepam, halazepam, lorazepam, oxazepam, and prazepam. Suitable 5-HT_{1A} receptor agonists or antagonists include buspirone, flesinoxan, gepirone and ipsapirone.

Generally, the compound of formula (I), or a salt thereof, and the additional therapeutic agents are formulated as separate dosage forms, where the separate dosage forms may be administered sequentially or separately at predetermined intervals and sequences, or administered simultaneously. Compound of formula (I) or salt thereof may also be administered together with the additional therapeutic agent in a single dosage form.

In yet another aspect, the present invention provides a pharmaceutical composition comprising a compound of formula I, or a pharmaceutically acceptable salt thereof, and a pharmaceutically acceptable excipient. The term “excipient” as used herein refers to any ingredient other than the compound of formula (I), salt thereof, and the other therapeutic agents. The term “excipient” also encompasses capsule shells, such as gelatin capsule shells, and tablet coatings. Examples of excipients include diluents, carriers, and stabilizers. The choice of excipients will depend on a variety of factors such as the particular mode of administration, the effect of the excipients on solubility and stability of the compound of formula (I) or salt thereof, and the nature of the dosage form. Descriptions of suitable pharmaceutically acceptable excipients and factors involved in their selection can be found in a variety of readily available sources, such as Remington's Pharmaceutical Sciences, 17^{th} ed., Mack Publishing Company, Easton, Pa., 1985.

Pharmaceutical compositions of the invention may be prepared in solid dosage forms, such as tablets, troches, lozenges, powders, or granules. Tablet dosage forms may contain a disintegrant, a binder, a diluent, lubricant, and other excipients. Examples of suitable disintegrants include sodium starch glycolate, sodium carboxymethyl cellulose, methyl cellulose, starch, and sodium alginate. Examples of suitable binders include microcrystalline cellulose, gelatin, polyethylene glycol, natural and synthetic gums, hydroxypropyl cellulose, and hydroxypropyl methylcellulose. Examples of suitable diluents include lactose (monohydrate, spray-dried monohydrate, anhydrous and the like), mannitol, xylitol, dextrose, sucrose, sorbitol, microcrystalline cellulose, starch and dibasic calcium phosphate dihydrate. Examples of suitable lubricants include magnesium stearate, calcium stearate, zinc stearate, sodium stearyl fumarate, and mixtures of magnesium stearate with sodium lauryl sulphate. Components of a tablet dosage form may be blended together and then compressed directly or by roller to form tablets. Tablet blends or portions of blends may alternatively be wet-, dry-, or melt-granulated, melt congealed, or extruded before tableTting. The final formulation may comprise one or more layers and may be coated or uncoated. More information on formulation of tablets can be found in Pharmaceutical Dosage Forms: Tablets Vol. 1, by H. Lieberman and L. Lachman (Marcel Dekker, New York, 1980).

Pharmaceutical compositions of the invention may also be prepared in liquid dosage forms, such as solution, suspensions, emulsion, syrups, and elixirs. Examples of excipients that may be used in a liquid dosage form include water, ethanol, polyethylene glycol, propylene glycol, methylcellulose, or a suitable oil. Emulsifying agents and suspending agents may also be employed in a liquid dosage form. Liquid dosage forms may also be prepared by the reconstitution of a solid, for example, from a sachet. Liquid dosage forms for parenteral administration may be prepared as aqueous solutions, sterile non-aqueous solutions, or as a dried form to be used in conjunction with a suitable vehicle such as sterile, pyrogen-free water. The preparation of parenteral formulations under sterile conditions, for example, by lyophilisation, may readily be accomplished using standard pharmaceutical techniques known in the art.

The relative amount of the compound in a pharmaceutical composition can very within wide limits, but is generally within the range of about 5% to about 95% by weight in solid composition and from about 5% to about 70% by weight in a liquid composition. Exemplary tablets may contain up to about 80% drug, about 10 weight % to about 90 weight % binder, up to about 85 weight % diluent, about 2 weight % to about 10 weight % disintegrant, and from about 0.25 weight % to about 10 weight % lubricant.

### EXAMPLES

The following examples are provided for the sole purpose of illustrating certain embodiments of the invention; they should not be construed as limiting the scope of the invention in any way.

### Example 1A

### Preparation of 7-(4-{4-[3-chloro-2-(trifluorometyl)phenyl]piperazin-1-yl}butoxy)-[1,8]-naphthyridin-2(1H)-one

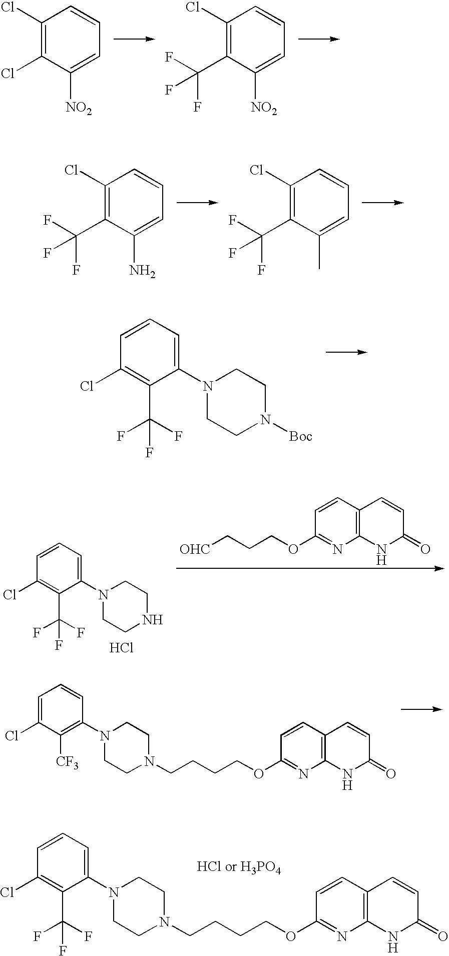

### 1-Chloro-3-nitro-2-trifluoromethyl-benzene

To a 5 L multineck flask equipped with a mechanical stirrer, a thermowell, a condenser, a nitrogen inlet line and a dropping funnel was added 2,3-dichloronitrobenzene (235.95 g, 1.229 mol), copper dust (471.90 g, 7.427 mol), and N,N-dimethylacetamide (1.82 L). Dibromodifluoromethane (246 mL, 566.00 g, 2.698 mol) was added in a thin stream via addition funnel over ˜15 min with stirring while inner temperature rose from 18° C. to 24° C. The reaction mixture was heated at 100° C. inner temperature for 3 hours after which GC indicated complete disappearance of the starting material (tR=4.61 min) and formation of the product (tR=3.52 min). The mixture was allowed to cool down to room temperature overnight with stirring then diluted (EtOAc, 0.8 L) and filtered through a pad of Celite (˜200 g). The solids were washed (3×550 mL EtOAc), the combined filtrates were washed (water 3.15 L then 2 L; brine 2.5 L) and dried (Na₄SO₄). The water layers were sequentially reextracted (EtOAc, 2×1.5 L). The combined organic were dried (Na₂SO₄), and the volatiles were evaporated. The residue (267.0 g) was purified on 1.2 kg of silica gel (eluting with 4 L of cyclohexane, 4 L of 0.5% EtOAc/cyclohexane, 8 L of 1% EtOAc/cyclohexane, and 8 L of 2% EtOAc/cyclohexane). The fractions were analyzed by GC and those having more than 70% of the product were combined and the volatales were evaporated to give 165.8 g (60%) of 1-Chloro-3-nitro-2-trifluoromethyl-benzene (79.4% GC purity with ¹H and ¹⁹F NMR's consistent with the desired product and indicating presence of 10-20% of impurities). The product was of sufficient purity to be used in the next step without further purification. GG analysis was done on a DB5 Column, 30 meters, 0.32 mm id, 0.25 um film, 75-300° C., 15° C./min., Injector temperature: 275° C., Detector temperature 350° C.

### 3-Chloro-2-trifluoromethyl-phenylamine

To a 5 L multineck flask equipped with a mechanical stirrer, a thermowell, a condenser, a nitrogen inlet line and a dropping funnel was added 1-Chloro-3-nitro-2-trifluoromethyl-benzene (221.27 g, 0.981 mol), ethanol (denatured with ˜5% ea. MeOH and i-PrOH; 3.46 L), water (0.87 L), zinc dust (384.71 g, 5.885 mol) and anhydrous calcium chloride (108.88 g, 0.981 mol). After being heated at reflux overnight, GC indicated complete disappearance of the starting material (tR=3.52 min) and formation of the product (tR=3.25 min). The mixture was cooled (˜30° C.) and filtered through a pad of Celite (˜200 g). The solids were washed (2×500 mL EtOH), and the combined filtrates were concentrated. EtOAc (0.5 L) was added to the residue. The organic layer was washed (water, 2x; brine). The water layers were sequentially reextracted (2×0.5 L EtOAc). The organic layers were dried (Na₂SO₄), combined, and the volatiles were evaporated to give 175.31 g (91%) of the 3-Chloro-2-trifluoromethyl-phenylamine (80.9% GC purity with ¹H and ¹⁹F NMR's consistent with the desired product and indicating presence of 10-20% of impurities and 0.5 equiv of EtOAc, hydrogen spectrum). The 3-Chloro-2-trifluoromethyl-phenylamine was of sufficient purity to be used in the next step without further purification. GC analysis done on a DB5 Column, 30 meters, 0.32 mm id, 0.25 um film, 75-300° C., 15° C./min., Injector temperature: 275° C., Detector temperature: 350° C. MS: APCl: M+1: 196.1 (195.0)

### 1-Chloro-3-iodo-2trifluoromethyl-benzene

To a suspension of 3-Chloro-2-trifluoromethyl-phenylamine (121.61 g, 0.622 mol) in water (356 mL) was added 37% aqueous HCl portionwise allowing reaction temperature to rise to 42° C. The mixture was stirred for 30 minutes then cooled with ice/MeOH bath to 5° C. DCM (40 mL) was added and the mixture was stirred for 10 minutes. A solution of NaNO₂ (53.52 g, 0.776 mol) in water (99 mL) was added dropwise while keeping the temperature of the reaction mixture below 5° C. After complete addition, the reaction mixture was stirred for 30 minutes and a solution of NaI (110.98 g, 0.740 mol) in water (198 mL) was added dropwise while keeping the temperature of the reaction mixture below 5° C. After completion of the addition, the reaction mixture was stirred for 1 hour and 15 minutes, and DCM (0.79 L) was added followed by NaHSO₃(35.92 g) to discharge iodine color. The organic layer was washed (brine), and the water layer was sequentially reextracted (2×0.2 L DCM). The organic layers were dried (Na₂SO₄), combined, and the volatiles were evaporated. The residue (180.55 g) was purified on 0.8 kg of silica gel (elution with 4 L of cyclohexane). The fractions were analyzed by GC, and those having more than 80% of the product were combined, and the volatiles were evaporated. The liquid residue was placed in the freezer for 1 hour, after which the solids were filtered, washed (pentane) and air-dried to afford 78.84 g (41%) of the desired product as beige solid in several crops (97-98% GC purity with ¹H and ¹⁹F NMR's clean and consistent with the desired product). The mother liquors were purified on 0.8 kg of silica gel (eluting with 4 L of cyclohexane). The fractions were analyzed by GC, and those having more than 80% of the product were combined and the volatiles were evaporated. The liquid residue was placed in the freezer for 1 hour, and solids were filtered, washed (pentane), and air-dried to give an additional 31.56 g (17%) of the 1-Chloro-3-iodo-2-trifluoromethyl-benzene as white solid (96% GC purity with ¹H and ¹⁹F NMR's clean and consistent with the desired product). Also, additional product was obtained (14.30 g, 8%, 81% pure GC) from mother liquors and 28.82 g (50% by GC, froim the less pure fractions. GC analysis done on a DB5 Column, 30 meters, 0.32 mm id, 0.25 um film, 75-300° C., 15° C./min., Injector temperature: 275° C., Detector temperature: 350° C., Retention time: 3.88 minutes. MS: APCl: M+: 306.0 (305.9)

### 4-(3-Chloro-2-trifluoromethyl-phenyl)-piperazine-1-carboxylic acid tert-butyl ester

To argon purged suspension of Pd₂(dba)₈ (16.45 g, 17.96 mmol) in toluene (1.29 L) in a 3 L four-neck flask equipped with a mechanical stirrer, a thermowell, and a condenser was added (±)-BINAP (33.88 g, 54.42 mmol), t-BuONa (42.53 g, 442.61 mmol), Boc-piperazine (76.45 g, 410.45 mmol), and 1-Chloro-3-iodo-2-trifluoromethyl-benzene (110.07 g, 359.18 mmol). The reaction mixture was purged for an additional 30 minutes with argon and heated at reflux under argon overnight (inner temperature was 105-107° C.). GC and ¹⁹F NMR indicated complete consumption of the starting material. The reaction was cooled to room temperature and filtered through a pad of Celite and the solids were washed (DCM). The filtrates were washed (brine), dried (Na₂SO₄), and the volatiles were evaporated. The residue (191 g) was dissolved in DCM, adsorbed on 400 g of silica gel, coevaporated with heptane (3×) to near dryness and chromatographed on 0.85 kg of silica gel (eluting with 2 L of cyclohexane then 2% EtOAc/cyclohexane) until the title compound started eluting. The polarity of elutent was then increased (5% EtOAc/cyclohexane) until nearly all of the desired product was off the column. The polarity of the solvent was further increased to 10% EtOAc/cyclohexane to elute remainder of the compound along with some impurities. The fractions were analyzed by TLC, and those that appeared to be clean product were combined, and the volatiles were evaporated to give 59.92 g (46%) of the yellow solid that was of 100% pure by GC with ¹H and ¹⁹F NMR's clean and consistent with the desired product. Also, less pure fractions were combined to give 24.14 g (18%, 82% by GC) of product. GC analysis done on a DB5 Column, 30 meters, 0.32 mm id, 0.25 μm film, 75-300° C., 15° C./min., Injector temperature: 275° C., Detector temperature: 350° C., Retention time: 10.23 minutes. MS: APCl: M+1: 365.2 (364.1)

### 1-(3-Chloro-2-trifluoromethyl-phenyl)-piperazine, hydrochloride salt

4M HCl/dioxane (185 mL) was added to a magnetically stirred solution of 4-(3-Chloro-2-trifluoromethyl-phenyl)-piperazine-1-carboxylic, acid tert-butyl ester (59.90 g, 0.164 mol) in EtOAc (625 mL). The mixture was stirred overnight after which the volatiles were evaporated, and the residue was triturated (EtOAc/ether), filtered, washed (EtOAc/ether, ether 2×) and dried in the vacuum oven at 60° C. overnight to give 43.30 g (88%) of 1-(3-Chloro-2-trifluoromethyl-phenyl)-piperazine, hydrochloride salt as white solid (98.7% pure by HPLC with ¹H and ¹⁹F NMR's clean and consistent with the desired product). The compound melted at 242° C. (softened at 238-240° C.). MS: APCl: M+1: 265.1 (264.1) HPLC: Pinnacle II Column, C18 5 um, 150×4.6 mm; Mobile phases: A=0.1% TFA/ACN, B=0.1% TFA/H₂O; Gradient: 4 min hold at 5% A, then linear gradient from 5% A to 100% A in 20 min at 1.5 mL/min then 4 min hold; Retention time: 13.42 minutes. CHN Found: C, 43.53; H, 4.02; N, 9.16; Cl, 23.32 Calc: C, 43.87; H, 4.35; N, 9.30; Cl, 23.55.

### 7-{4-[4-(3-Chloro-2-trifluoromethyl-phenyl)-piperazin-1-yl]-butoxy}-1H-[1,8]naphthyridin-2-one

The 1-(3-Chloro-2-trifluoromethyl-phenyl)-piperazine hydrochloride salt (8.3 g, 27.6 mmol, 1.0 equiv), 4-(7-Oxo-7,8-dihydro-[1,8]naphthyridin-2-yloxy)-butyraldehyde (6.7 g, 28.7 mmol, 1.04 equiv) and triethyl amine (8.0 mL, 57.1 mmol, 2.07 equiv) were dissolved in CH₂Cl₂ (80 mL) and stirred 10 minutes. To the reaction mixture was added Na(OAc)₃BH (7.9 g, 37.2 mmol, 1.35 equiv) which caused a mild exothermic. After stirring 20 minutes, the reaction was quenched by addition of saturated aqueous NaHCO₃ (˜50 mL). The two phase mixture was stirred vigorously for 10 minutes followed by removal of the aqueous layer and concentration of the organic layer in vacuo. Methanol (10 mL) was added and the mixture was concentrated in vacuo. Methanol (˜80 mL) was added and the resulting mixture was heated to 50° C. The solution was cooled slowly to room temperature with seeding (crystals persisted at approximately 40° C.), allowed to stir overnight after which the solid was removed by filtration. The wet cake was dried in vacuum oven at 50° C. overnight to afford 7-{4-[4-(3-Chloro-2-trifluoromethyl-phenyl)-piperazin-1-yl]-butoxy}-1H-[1,8]naphthyridin-2-one (10.3 g, 78% yield, 99 3% pure by HPLC). ¹H NMR (400 MHz, CDCl₃-d) δ ppm 9.25 (s, 1 H) 7.69 (d, J=8.58 Hz, 1 H) 7.61 (d, J=9.36 Hz, 1 H) 7.32 (t, J=8.09 Hz, 1 H) 7.17 (d, J=7.80 Hz, 1 H) 7.10 (d, J=8.19 Hz, 1 H) 6.57 (d, J=8.58 Hz, 1 H) 6.50 (d, J=9.55 Hz, 1 H) 4.37 (t, J=6.43 Hz, 3 H) 2.44-2.49 (m, 2 H) 2.94-3.00 (m, 5 H) 1.79-1.86 (m, 2 H) 1.64-1.72 (m, 2 H)

### 7-{4-[4-(3-Chloro-2-trifluoromethyl-phenyl)-piperazin-1-yl]-butoxy}-1H-[1,8]naphthyridin-2-one, hydrochloride salt

To a flask, containing the 7-{4-[4-(3-Chloro-2-trifluoromethyl-phenyl)-piperazin-1-yl]-butoxy}-1H-[1,8]naphthyridin-2-one, was added enough THF to dissolve the compound (˜3 mL). A 1N HCl solution in ether was added (0.68 mL). A precipitate formed immediately. This was swirled and sonicated then was let to sit for ˜15 min and then the solid was collected by filtration then dried at ˜50° C. in a vacuum oven overnight. This resulted in 320 mg of 7-{4-[4-(3-Chloro-2-trifluoromethyl-phenyl)-piperazin-1-yl]-butoxy}-1H-[1,8]naphthyridin-2-one, hydrochloride salt as an off-white solid. MS: APCl: M+1: 481.2 (480.2). HPLC: Chromolith column, C18, 100×4.6 mm, Mobile Phase—5:95 to 95:5 ACN/water (w/0.1% TFA) over 10 min then hold for 5 min, 1.5 mL/min, Wavelength—254, % Purity—97.0, Retention Time—7.512 min. CHN Found: C, 52.00; H, 4.92; N, 10.40. This calculates for C₂₃H₂₄ClF₃N₄O₂ 1.27 HCl; ¹H NMR (400 MHz, DMSO-d₆) δ 11.96 (s, 1 H) 10.64 (s, 1 H) 7.97 (d, J=8.4 Hz, 1 H) 7.81 (d, J=9.4 Hz, 1 H) 7.60 (t, J=8.2 Hz, 1 H) 7.42 (d, J=8.0 Hz, 1 H) 7.35 (d, J=8.4 Hz, 1 H) 6.62 (d, J=8.4 Hz, 1 H) 6.34 (d, J=9.4 Hz, 1 H) 4.34 (t, J=6.1 Hz, 2 H) 3.54 (d, J=11.7 Hz, 2 H) 3.19 (m, 6 H) 3.08 (m, 2 H) 1.77 (m, 4 H)

### 7-{4-[4-(3-Chloro-2-trifluoromethyl-phenyl)-piperazin-1-yl]-butoxy}-1H-[1,8]naphthyridin-2-one, phosphate salt

The 7-{4-[4-(3-Chloro-2-trifluoromethyl-phenyl)-piperazin-1-yl]-butoxy}-1H-[1,8]naphthyridin-2-one (10.2 g, 21.2 mmol) was dissolved in MeOH at 58° C. (100 mL). In a separate vessel, the H₃PO₄ (2.18 g, 22.3 mmol, 1.05 equiv) was dissolved in methanol (10 mL). The phosphoric acid solution was added dropwise to free base solution over approximately 1 minute, and the salt began crystallizing before all phosphoric acid had been added in a mildly exothermic crystallization. The slurry was cooled slowly to room temperature and stirred overnight. The slurry was filtered and vacuum dried overnight to furnish the desired salt (9.4 g, 77% yield, 98.6% pure by HPLC). ¹H NMR (400 MHz, DMSO-d₆) δ 7.96 (d, J=8.59 Hz, 1 H), 7.82 (d, J=9.37 Hz, 1 H), 7.56 (t, J=8.20 Hz, 1 H), 7.30-7.39 (m, J=17.48, 8.10 Hz, 2 H), 6.63 (d, J=8.40 Hz, 1 H), 6.35 (d, J=9.57 Hz, 1 H), 4.34 (t, J=6.25 Hz, 2 H), 3.49 (s, 10 H), 3.01 (s, 4 H), 1.63-1.80 (m, 4 H).

### Example 1B

### Preparation of 7-(4-{4-[3-chloro-2-(trifluoromethyl)phenyl]piperazin-1-yl}butoxy)-[1,8]-naphthyridin-2(1H)-one

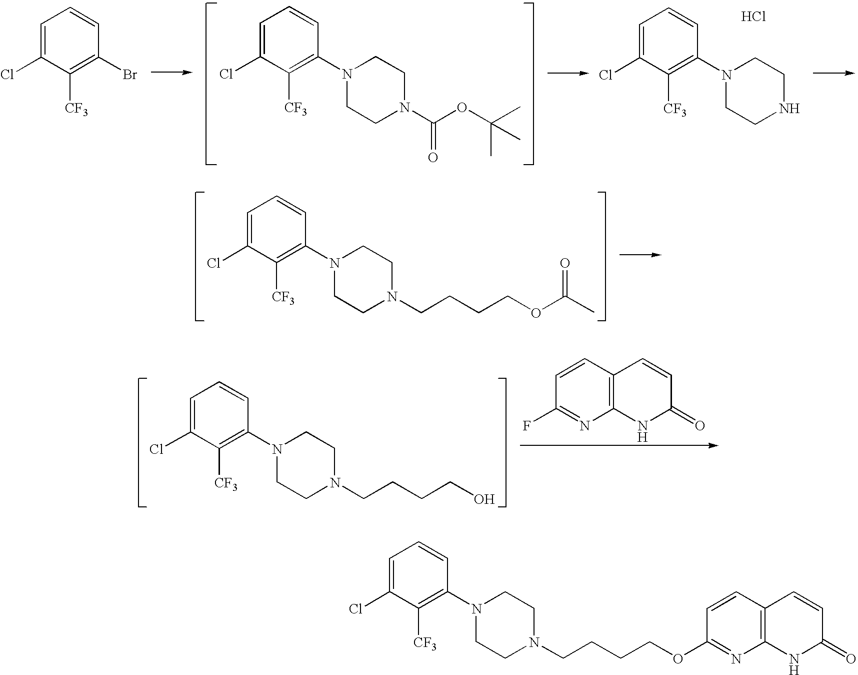

### 1-(3-Chloro-2-trifluoromethyl-phenyl)-piperazine hydrochloride

To a degassed solution of toluene (200 mL) containing racemic-2,2′-bis(diphenylphosphino)-1,1′-binaphthyl (490 mg, 0.77 mmoles), Pd₂(dba)₃ (291.09 mg, 0.308 mmoles). N-Boc piperazine (28.71 g, 154.17 mmoles) and sodium tert-amylate (20.4 g, 185 mmoles) was added aryl bromide (40.0 g, 154.17 mmoles). The reaction was heated to 90° C. and stirred at that temperature for 17 hours. The reaction was cooled to approximately room temperature and washed with water (100 mL). To the organic layer was added hydrogen chloride (1.5 equiv; 231.26 mmoles; 17.52 mL; 19.27 g) and the reaction was heated to 80° C. (approximately 3 hours). The reaction was equipped with a Dean-Stark apparatus and water removed by atmospheric distillation until <0.1% water remained. The reaction was cooled to room temperature and stirred for two days. The resultant slurry was filtered, and the wet cake washed with toluene to provide 52.6 g (113% crude yield) of the product, which was sufficiently pure to use without further purification.

### Acetic acid 4-[4-(3-chloro-2-trifluoromethyl-phenyl)-piperazin-1-yl]-butyl ester

A slurry of 1-(3-Chloro-2-trifluoromethyl-phenyl)-piperazine hydrochloride (46.4 g, 154 mmoles) in toluene (464 mL) was washed with aqueous potassium hydroxide (12.98 g, 231 mmoles in 100 mL of water). To this solution was added triethylamine (31.20 g, 42.98 mL, 308.4 mmoles) and 4-bromo-1-butanol acetate (26.93 mL, 36.09 g, 185.0 mmoles). The whole was heated to 100° C. and stirred until all starting material had been consumed (˜18 hours). The crude reaction mixture was used directly in the next reaction.

### 4-[4-(3-Chloro-2-trifluoromethyl-phenyl)-piperazin-1-yl]-butan-1-ol

To the preceding crude reaction mixture was added potassium hydroxide (25.95 g, 462.5 mmoles) in MeOH (100 mL) which produced an exotherm. Upon cooling, the reaction was heated to distill off the methanol and reach an internal temperature of 115° C. (added toluene 2×50 mL to maintain liquidity). Upon cooling to room temperature, the reaction was extracted with water (100 mL). The resultant mixture was distilled until the internal temperature reached 105° C. (0.4% water). The reaction was cooled to 50° C. and used directly in the next reaction.

### 7-{4-[4-(3-Chloro-2-trifluoromethyl-phenyl)-piperazin-1-yl]-butoxy}-1-[1,8]naphthyridin-2-one

To the above solution was added 7-Fluoro-1H-[1,8]naphthyridin-2-one (25.31 g, 154.18 mmoles, for procedure see below) followed by sodium tert-amylate (33.96 g, 308.36 mmoles) which precipitated an exotherm (50° C. to 83° C.). When the reaction had cooled to 63° C. additional sodium tert-amylate (3.40 g, 30.84 mmoles) was added and the reaction was stirred overnight. The reaction was cooled to approximately 30° C. and quenched with water (100 mL). The aqueous phase was removed and the organics concentrated. The residue afforded the title compound as a solid upon exposure to IPA water (35 g, 39% yield). ¹H NMR (400 MHz, CDCl₃-d) δ ppm 9.25 (s, 1 H) 7.69 (d, J=8.58 Hz, 1 H) 7.61 (d, J=9.36 Hz, 1 H) 7.32 (t, J=8.09 Hz, 1 H) 7.17 (d, J=7.80 Hz, 1 H) 7.10 (d, J=8.19 Hz, 1 H) 6.57 (d, J=8.58 Hz, 1 H) 6.50 (d, J=9.55 Hz, 1 H) 4.37 (t, J=6.43 Hz, 3 H) 2.44-2.49 (m, 2 H) 2.94-3.00 (m, 5 H) 1.79-1.86 (m, 2 H) 1.64-1.72 (m, 2 H)

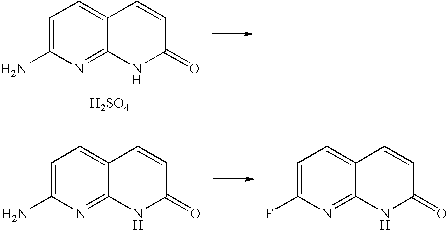

### 7-Amino-1H-[1,8]naphthyridin-2-one

7-Amino-1H-[1,8]naphthyridin-2-one sulfate (797 g, 3.08 mole) was slurried in water (5 L). The pH of the suspension was adjusted to >10 by the gradual addition of 6N aqueous sodium hydroxide with cooling via an ice bath. The slurry was vigorously stirred for ˜30 min then filtered via suction filtration. The filter cake was suspended in water (4 L) and then filtered. The water wash was repeated 3× to remove all salts. The filter cake was dried in vacuo at 45° C. to give 7-Amino-1H-[1,8]naphthyridin-2-one (493 g, 3.06 mole, 99%) as a yellow solid.

### 7-Fluoro-1H-[1,8]naphthyridin-2-one

7-Amino-1H-[1,8]naphthyridin-2-one (290 g, 1.81 mole) was added to a nalgene reactor containing HF pyridine (70%, 1.25 Kg) with cooling via an ice bath. The solution was stirred with a nalgene stir shaft and paddle. Sodium nitrite (150 g, 2.17 more) was added to the reaction carefully in small portions with salt ice bath cooling and vigorous stirring. A vigorous reaction occurred with each addition. The reaction exothermed considerably and evolution of gas was vigorous. After all of the sodium nitrite was added the reaction was vigorously stirred with ice bath cooling under nitrogen. Solids form and the reaction was sampled after 30 min. The reaction was judged complete by ¹H NMR at this time. The reaction was carefully diluted to a volume of approximately 4 L with ice/water. The suspension was suction filtered and the solids washed by resuspending in water (4 L) and suction filtering (3×). The solid material was then resuspended in ethyl acetate (4 L) and suction filtered (2×). The resulting bright yellow filter cake was pooled with another batch (prepared from 190 g of 7-Amino-1H-[1,8]naphthyridin-2-one by a virtually identical procedure and similar stoichiometry of reagents). The pooled material was dried in vacuo to give 7-Fluoro-1H-[1,8]naphthyridin-2-one (478 g, 2.91 mole, 97% yield) as a bright yellow solid. MS: API-ES: M+1: 165.2 (164.04). ¹H NMR (400 MHz, DMSO-d₆) δ 12.34 (s, 1 H), 8.31 (t, J=8.20 Hz, 1 H), 7.97 (d, J=9.67 Hz, 1 H), 7.00 (dd, J=8.20, 2.05 Hz, 1 H), 6.58 (d, J=9.37 Hz, 1 H) CHN Found: C, 58.06; H, 2.89; N, 17.03; F, 11.58; Calc: C, 58.41; H, 3.09; N, 17.03; F, 11.54

### Example 2

### Biological Assays

Certain pharmacological properties of the compound of formula (I) or a salt thereof were investigated using the assays described below. The tern “test compound” as used in the description of the assays refers to the HCl salt of compound of formula (I). The result of each assay is presented in Table 1.

1) Dopamine D₂ Receptor Binding Assay

[³H]Spiperone binding to a membrane preparation from CHO-hD2L cells was carried out in 250 μL of 50 mM Tris-HCl buffer containing 100 mM NaCl, 1 mM MgCl₂ and 1% DMSO at pH 7.4. Duplicate samples containing (in order of addition) the test compound, 0.4 nM [³H]spiperone and approximately 12 μg protein were incubated for 120 min at room temperature. Bound radioligand was separated by rapid filtration under reduced pressure through Whatman GF/B glass fiber filters previously treated with 0.3% polyethyleneimine (PEI). Radioactivity retained on the filter was determined by liquid scintillation spectrophotometry. Specific binding determined in the presence of 1 mM haloperidol was 95%.

2) Serotonin 1A (5-HT_{1A}) Receptor Binding Assay

The assay was conducted in membranes prepared from HeLa cells transfected with the cDNA for h5-HT_{3A} receptors using [³H] 8-hydroxy-2-(di-n-propylamino)-tetraline ([³H] 8-OH-DPAT, final concentration 2.0 nM) as reference 5-HT_{3A} receptor agonist. Assay buffer consisted of 50 mM Tris-HCl with 10 mM MgSO₄, 0.5 mM EDTA and 0.1% ascorbic acid, at pH 7.4 (250 μL total volume). Triplicate samples containing (in order of addition) the test compound, 100× in 100% DMSO, [³H] 8-OH-DPAT and cell homogenate was incubated for 2 hr at room temperature. Experiments were terminated by rapid filtration through Whatman GF/B glass fiber filters soaked in assay buffer with 0.3% PEI by washing three timnes with 1 mL of wash buffer (50 mM Tris-HCl, pH 7.4) using a Brandel MLR-96T cell harvester. Specific binding (85%) was determined in the presence of 10 μM 8-OH-DPAT.

3) [³H]Thymidine Uptake Assay for Dopamine D₂ Intrinsic Activity

The assay was conducted using CHO pro-5 cells containing the D₂ receptor and conventional 96-well sterile plates. Serum was removed from the cells by washing the cells twice with 200 μL of serum-free media. 90 μL serum-free media was added to each well. The plates ware incubated for two to three hours. 10 μL of serum-containing media, as a positive control, vehicle (serum-free media), antagonist control (haloperidol), or the test compound, and standards (10 μL of a 10 μM solution for a final concentration of 1 μM) in serum-free media were added to wells. The plates were returned to the incubator. Eighteen hours later [³H]thymidine was added (0.5 μCi/well in 10 μL of serum-free media) and the plates were returned to the incubator. Four hours later trypsin (0.25%) was added (100 μL/well). The plates were returned to the incubator, once again. One hour later the assay was terminated by rapid filtration through Whatman GF/C glass fiber filters. Filters were washed four times with 500 mL of 50 mM Tris-HCl pH 7.0 buffer, for example, using a Brandel MLR-96T cell harvester. Radioactivity remaining on the filters was estimated with a Wallac 1205 Betaplate liquid scintillation counter (50% efficiency). Intrinsic activity is defined as total uptake (1 μM Quinpirole) minus serum-free media (no uptake). The test compounds was compared to 1 μM Quinpirole (full dopamine D₂ receptor agonist), which was classified as 100% intrinsic activity.

4) [³H]Thymidine Uptake Assay for Serotonin 1A Intrinsic Activity

CHO p-5 cells transfected with h5-HT_{1A} cDNA were seeded into 96-well plates at a density of approximately 5×10⁸ cells/well. These were grown for 3 days at 37° C. in an incubator with alpha minimum essential medium (αMEM) and 10% fetal calf serum containing penicillin (100 U/mL) and streptomycin (100 μg/mL). The wells were then rinsed by washing twice with 200 μL of serum-free media, and 90 μL serum-free media was added to each well. The plates were incubated for two to three hours, then 10 μL of serum-containing media (positive control), vehicle (10 μL DMSO), negative control (10 μL of an antagonist in DMSO) or different concentrations of the test compound and standards (10 μL DMSO solution) in serum-free media were added to appropriate wells. The plates were incubated for 18 hrs. Then [³H]thymidine was added (0.5 μCi/well in 10 μL of serum-free media) after which the plates were returned to the incubator. Four hours later, trypsin (0.25%) was added (100 μL/well), and plates were returned again to the incubator. The assay was terminated 1 hr later by rapid filtration through Whatman GF/C glass fiber filters. Filters were washed three times with 500 μL of 50 mM Tris-HCl buffer at pH 7.0 using a Brandel MLR-96T cell harvester. Radioactivity remaining on the filters was estimated with a Wallac 1205 Betaplate liquid scintillation counter (50% efficiency). Percent response was defined as total uptake (1 μM (+)-8-OH-DPAT) minus serum-free media (no uptake). The test compound was compared to 1 μM (+)-8-OH-DPAT (full 5-HT_{1A} agonist), which was classified as 100% response for 5-HT1A. All assays were performed in quadruplicate with each compound occupying one full column (8 wells) per plate.

TABLE 1
Results of Biological Assays

| Assay Name | Results |
|---|---|
| Dopamine D₂ Receptor Binding Assay (Ki, nM) | 3.9 |
| Serotonin 1A (5-HT_{1A}) Receptor Binding Assay (Ki, nM) | 4 |
| [³H]Thymidine Uptake Assay for Dopamine D₂ Intrinsic | 55 |
| Activity | |
| [³H]Thymidine Uptake Assay for Serotonin 1A Intrinsic | 90 |
| Activity (%) | |

What is claimed is:

1. A compound of formula of formula (I) 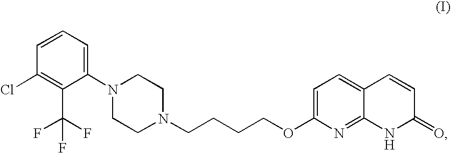
or a pharmaceutically acceptable salt thereof.

2. A pharmaceutically acceptable salt according to claim 1.

3. A pharmaceutically acceptable salt according to claim 2, which is a salt derived from an acid selected from hydrochloric, hydrobromic, nitric, carbonic, monohydrogencarbonic, phosphoric, monohydrogenphosphoric, dihydrogenphosphoric, sulfuric, monohydrogensulfuric, hydriodic, phosphorous acids, acetic, adipic, ascorbic, aspartic, benzenesulfonic, benzoic, besylic, camsylic, cholic, citric, malic, maleic, malonic, mandelic, methanesulfonic, succinic, sulphosalicylic, or tryptophanic acid.

4. The pharmaceutically acceptable salt according to claim 3, which is a salt derived from hydrochloric acid or phosphoric acid.

5. A pharmaceutical composition, comprising:
(a) a compound or a pharmaceutically acceptable salt thereof according to claim 1, and
(b) a pharmaceutically acceptable excipient.

6. A pharmaceutical composition, comprising:
(a) a compound or a pharmaceutically acceptable salt thereof according to claim 1, and
(b) a second therapeutic agent

7. The pharmaceutical composition according to claim 6, wherein the second therapeutic agent is an antidepressant or anti-anxiety agent.

8. The compound or pharmaceutically acceptable salt thereof according to claim 1 for use as a medicament.

9. The pharmaceutical composition according to any of claims 5-7 for use as a medicament.

10. A method of treating a central nervous system disorder in a mammal, comprising administering to the mammal a therapeutically effective amount of a compound or a pharmaceutically acceptable salt thereof according to any of claims 1-3.

11. The method according to claim 10, wherein the mammal is a human.

12. The method according to claim 11, wherein the central nervous system disorder is selected from the group consisting of: major depression, single episode depression, recurrent depression, child abuse induced depression, postpartum depression, dysthymia, cyclothymia, bipolar disorder, delusional disorder, substance-induced psychotic disorder, brief psychotic disorder, shared psychotic disorder, psychotic disorder due to a general medical condition, autism, pervasive development disorder, attention deficit hyperactivity disorder, generalized anxiety disorder, panic disorder, obsessive-compulsive disorder, post-traumatic stress disorder, phobias, schizophrenia, schizophrenia with concomitant depression, schizophrenia with concomitant anxiety, and schizoaffective disorder.

13. The method according to claim 12, wherein the central nervous system disorder is schizophrenia or bipolar disorder.

14. A method of treating a disorder in a mammal, comprising administering to the mammal:
(a) an effective amount of a compound or a pharmaceutically acceptable salt thereof according to claim 1, and
(b) an therapeutically effective amount of an antidepressant or an anti-anxiety agent,
wherein the disorder is selected from the group consisting of: major depressive disorders, dysthymic disorders, depressive neurosis and neurotic depression, melancholic depression, atypical depression, bipolar disorder, cyclothymic disorder, conduct disorder, disruptive behavior disorder, attention deficit hyperactivity disorder, anxiety disorders, borderline personality disorder, schizophrenia, neurodegenerative disorders, movement disorders, dyskinesias, chemical dependencies and additions, behavioral addictions, and ocular disorders.

15. Use of a compound or a pharmaceutically acceptable salt thereof according to claim 1 for the manufacture of a medicament for the treatment of a central nervous system disorder.
